(19) United States
(12) Patent Application Publication
(10) Pub. No.: US 2010/0094082 A1
(43) Pub. Date: Apr. 15, 2010

(54) MEDICAL EXAMINING DEVICE WITH AN ANGULARLY OFFSET FIBER OPTIC CHANNEL

(76) Inventors: Jay Iinuma, Sierra Madre, CA (US); Clifford Yamamoto, Torrance, CA (US)

Correspondence Address: STETINA BRUNDA GARRED & BRUCKER, 75 ENTERPRISE, SUITE 250, ALISO VIEJO, CA, 92656, US

(21) Appl. No.: 12/248,634

(22) Filed: Oct. 9, 2008

(51) Int. Cl. A61B 1/303

(52) U.S. Cl. 600/105

(57) ABSTRACT

There is provided a medical examining device including an hollow sample tube defining a tube first end and an opposing tube second end. The sample tube includes a tube wall extending between the tube first and second ends. The tube wall includes tube inner and outer surfaces extending between the tube first and second ends. The tube inner surface defines a central sampling channel configured to longitudinally receive a sampling tool. A longitudinal channel partially extends through the tube wall between the tube first end towards the tube second end along an optical axis. The optical axis is angularly offset and extends toward the sampling channel axis adjacent the tube second end. A translucent lens is positioned in optical communication with the longitudinal channel adjacent the tube second end. A fiber optic viewing device is disposed within the longitudinal channel and is in optical communication with the translucent lens.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

1. Field of the Invention

The present invention relates generally to a medical examining device and more specifically to a medical examining device configured to examine a body cavity of a patient with a fiber optic viewing tool.

2. Description of the Related Art

Regular medical examinations are a recommended practice for living a healthy life. For women, it is important to have routine gynecological examinations. One of the most critical conditions tested during gynecological examinations is cervical cancer. The gynecologist may conduct a safe and inexpensive test to test for cervical cancer. The test is commonly referred to as a Papanicolaou smear, or Pap test. Although the Pap test is safe and inexpensive, many women forgo routine testing because of the uncomfortable nature of the examination. By forgoing routine gynecological examinations, gynecological problems may advance to an untreatable state.

A Pap test typically requires a sample from the patient's cervix. The sample may be a tissue sample or a mucous sample. The sample is generally applied to a slide and analyzed for cancerous abnormalities. In addition, the gynecological examination may include a visual examination of the vaginal cavity for discoloration and/or growths, particularly if the patient has a history of cancer in their family.

Access to the cervix is typically achieved with the assistance of a rigid metal speculum. The speculum typically defines a duckbill-like configuration that moves between open and closed positions. The speculum is placed in a closed position and is inserted into the vaginal cavity. Once inserted, the speculum is opened to spread the vaginal cavity to provide access to the patient's cervix.

The use of the speculum tends to be very uncomfortable for a patient, both mentally and physically. The appearance of the speculum may be very intimidating to a patient, and immediately make the patient apprehensive about the procedure. Furthermore, speculums are commonly fabricated out of a metal material, which creates a cold, uncomfortable sensation during contact with the patient.

Recent advancements in technology have provided less invasive tools for viewing inside a patient's body cavity. For instance, fiber optic viewing tools may be inserted into a patient's body cavity for viewing the body cavity. The fiber optic viewing tools tend to have a small diameter, which makes the examination more tolerable for the patient.

With regard to gynecological examinations, it would be desirable to employ the use of fiber optic viewing tools to allow a gynecologist to view the vaginal cavity while performing the Pap test. However, as previously mentioned, gynecological examinations typically require a patient sample for testing and analysis; therefore, a separate sampling tool may be required to acquire a sample for testing. Furthermore, even if a separate sampling tool is used with the fiber optic viewing tool, the fiber optic viewing tool may provide a field-of-view that does not include the area where the sampling tool acquires a patient sample. In other words, it may be difficult to align the fiber optic viewing tool with the sampling tool, especially since there is very limited space within the patient cavity.

Moreover, use of a fiber optic viewing tool during gynecological examinations may require thorough cleansing and sanitation of the viewing tool between uses on different patients. The sanitation procedure for the fiber optic viewing tool may be very time consuming and expensive, making it commercially impractical.

As is apparent from the foregoing, there exists a need in the art for an improved medical examining device configured for use with a fiber optic viewing tool for examining a body cavity of a patient. The present invention addresses this particular need, as will be discussed in more detail below.

### BRIEF SUMMARY OF THE INVENTION

According to an aspect of the present invention, there is provided a medical examining device for use with a tissue sampling tool. The medical examining device includes an elongate, hollow sample tube defining a tube first end and an opposing tube second end. The sample tube includes a tube wall extending between the tube first end and the tube second end. The tube wall includes a tube inner surface and a tube outer surface extending between the tube first end and the tube second end. The tube inner surface defines a central sampling channel extending along a sampling channel axis. The central sampling channel is sized and configured to longitudinally receive the sampling tool along the sampling channel axis. The medical examining device also includes a longitudinal channel extending partially through the tube wall from the tube first end towards the tube second end along an optical axis. The optical axis is angularly offset and extends toward the sampling channel axis adjacent the tube second end. The medical examining device further includes a translucent lens positioned in optical communication with the longitudinal channel adjacent the tube second end. A fiber optic viewing device is disposed within the longitudinal channel and is in optical communication with the translucent lens.

The medical examining device may allow a doctor to more easily examine a body cavity using a fiber optic viewing device. In particular, the image receiving end of the fiber optic viewing device may be directed toward the examination area by the angular offset of the longitudinal channel. In other words, the angular offset may cause an extension of the optical axis to intersect with a sample channel axis defined by the sample channel. In this manner, the doctor may focus on the examination area of the body cavity without removing the tissue sampling tool from the field of view.

The medical examining device may include an inflatable cuff disposed about the sample tube. The inflatable cuff may be inflated after insertion into the body cavity to open the body cavity for examination.

The medical examining device may also be used in connection with a central unit. The central unit may include light source for illuminating the examination area of the body cavity. The medical examining device may include a light receiving surface disposable in optical communication with the light source to receive light therefrom. The medical examining device may be formed of a translucent material to allow light received from the light source to pass along the sample tube to emit from the tube second end. The central unit may also include a fluid source for inflating the inflatable cuff.

The present invention is best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings in which like numbers refer to like parts throughout and in which:

FIG. 1 is an exploded top perspective view of a medical examination assembly including a medical examining device and a central unit;

FIG. 2 is a side elevation view of the medical examination assembly illustrated in FIG. 1;

FIG. 3 is an end view of the medical examining device including a central sampling channel, a longitudinal channel and a pair of retention flanges;

FIG. 4 is a partial lower elevation view of the medical examining device illustrated in FIG. 3, the medical examining device including a fluid port and a light receiving surface;

FIG. 5 is a side sectional view of the medical examining device illustrated in FIGS. 3 and 4, the central sampling channel extending between a tube first end and a tube second end, the central sampling channel defining a sampling channel axis and the longitudinal channel defining an optical axis;

FIG. 6 is a partial, enlarged view of the tube second end of the medical examining device illustrated in FIG. 5, the medical examining device including a translucent lens disposed adjacent the tube second end in optical communication with the longitudinal channel, the optical axis being angularly offset towards the sample channel axis adjacent the tube second end;

FIG. 7 is an upper perspective view of the medical examination assembly, the medical examining device being disengaged with the central unit, and an inflatable cuff being disengaged from the medical examining device;

FIG. 8 is an upper perspective view of the medical examination assembly, the medical examining device being engaged with the central unit, and the inflatable cuff being disposed on the medical examining device;

FIG. 9 is an upper elevation view of the central unit illustrated in FIGS. 7 and 8, the central unit including a pair of retention members and a fluid port;

FIG. 10 is side sectional view of the central unit illustrated in FIG. 9;

FIG. 11 is side view of the medical examination assembly inserted into the vaginal cavity of a patient, the inflatable cuff being in a deflated position; and

FIG. 12 is a side view of the medical examination assembly illustrated in FIG. 11, the inflatable cuff being in an inflated position.

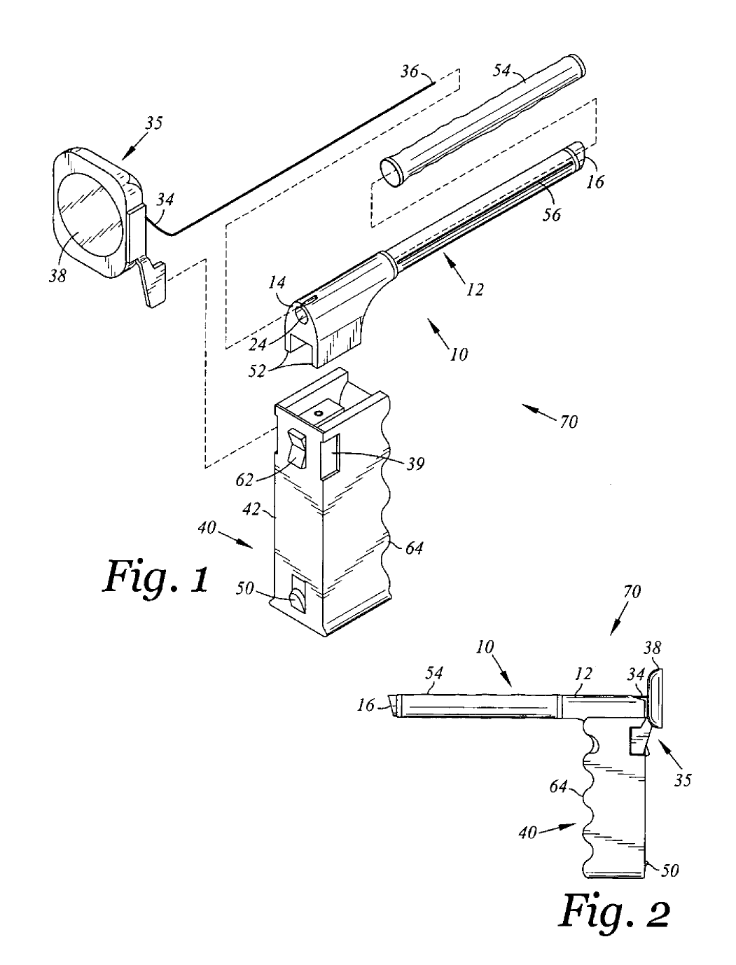

### DETAILED DESCRIPTION OF THE INVENTION

Referring now to the drawings wherein the showings are for purposes of illustrating a preferred embodiment of the present invention only, and not for purposes of limiting the same, there is shown a medical examining device 10 constructed in accordance with the present invention. According to various aspects of the present invention, the medical examining device 10 is sized and configured to facilitate the use of fiber optic viewing technology during a medical examination of a patient's body cavity 4, such as a vaginal cavity during a Papanicolaou smear, or Pap test.

It is understood that examination of a body cavity 4 oftentimes requires visual examination by a doctor, as well as a patient sample for testing and analysis. Accordingly, the medical examining device 10 is configured for use with a viewing tool 35 and a tissue sampling tool 68. The viewing tool 35 includes a fiber optic viewing device 34 having an image receiving end 36 configured to receive images, similar to a photo camera or a video camera. In this manner, the image receiving end 36 is disposed within the body cavity 4 adjacent a desired examination area in a direction facing the examination area to enable a doctor to view the examination area. The viewing tool 35 further includes a display screen 38 connected to the fiber optic viewing device 34 to display the images received by the image receiving end 36. In this manner, images are transferred along the length of the fiber optic viewing device 34 between the image receiving end 36 and the display screen 38.

As used herein, a tissue sampling tool 68 refers to a medical instrument configured to engage with a patient 2 for purposes for obtaining a patient sample for diagnostic analysis and/or testing. The patient sample may include a mucous sample, tissue sample, or another collection of one or more cells for purposes of testing and analysis. Accordingly, the tissue sampling tool 68 may be configured to gently wipe the body cavity walls for obtaining a mucus sample. Alternatively, the tissue sample tool 68 may be configured to extract a portion of the body cavity wall for obtaining a tissue sample.

According to one embodiment, the medical examining device 10 includes an elongate, hollow sample tube 12 that is detachably connectable to the viewing tool 35. The sample tube 12 defines a tube first end 14 and an opposing tube second end 16. The tube second end 16 is positionable within the patient's body cavity 4 during the medical examination. As such, the sample tube 12 may include rounded corners 25 to provide a more comfortable insertion of the sample tube 12 into the body cavity 4. The sample tube 12 includes a tube wall 18 extending between the tube first end 14 and the tube second end 16. The tube wall 18 includes a tube inner surface 20 and a tube outer surface 22 extending between the tube first end 14 and the tube second end 16.

The tube inner surface 20 defines a central sampling channel 24 extending through the sample tube 12. The central sampling channel 24 extends along a sampling channel axis 26. The central sampling channel 24 is sized and configured to facilitate insertion of the tissue sampling tool 68 into the body cavity 4 when the sample tube 12 is inserted into the body 4. In particular, the central sampling channel 24 longitudinally receives the tissue sampling tool 68. The tissue sampling tool 68 is inserted into the central sampling channel 24 along the sampling channel axis 26 when the sample tube 12 is inserted into the body cavity 4. As depicted in the figures, the sample tube 12 and central sampling channel 24 are substantially cylindrical in nature. However, it is understood that the sample tube 12 and central sampling channel 24 may define other shapes and configurations without departing from the spirit and scope of the present invention.

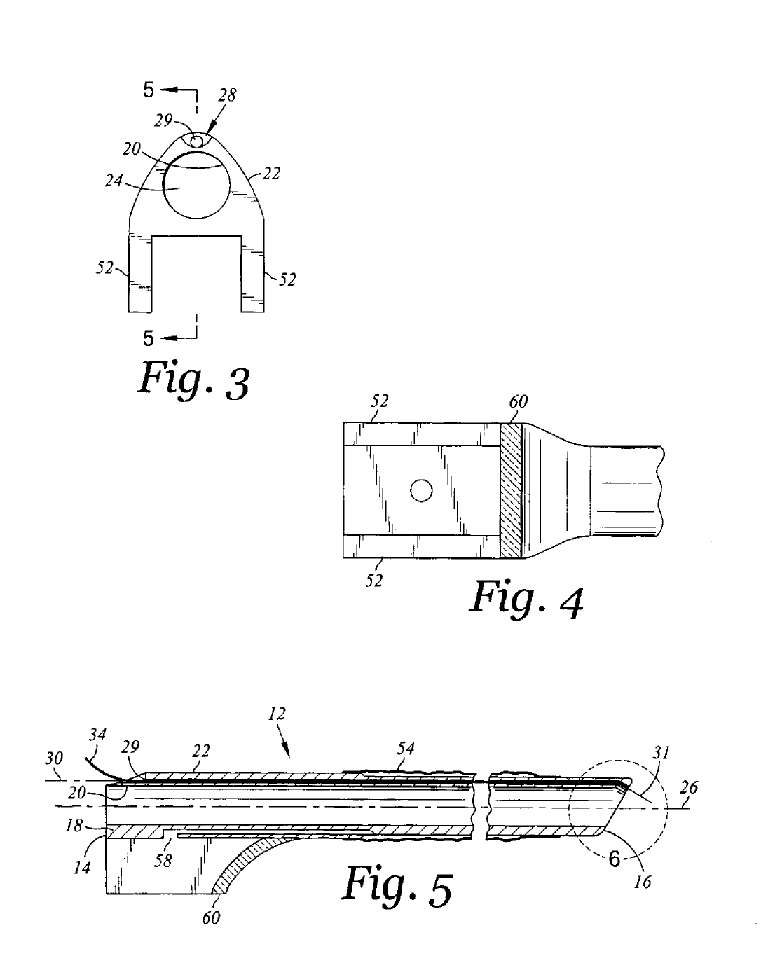

The medical examining device 10 further includes a longitudinal channel 28 extending partially through the tube wall 18 between the tube first end 14 and the tube second end 16. In this regard, the longitudinal channel 28 does not extend completely between the tube first end 14 and the tube second end 16. The longitudinal channel 28 includes an opening 29 disposed adjacent the tube first end 14, as best depicted in FIGS. 3 and 5.

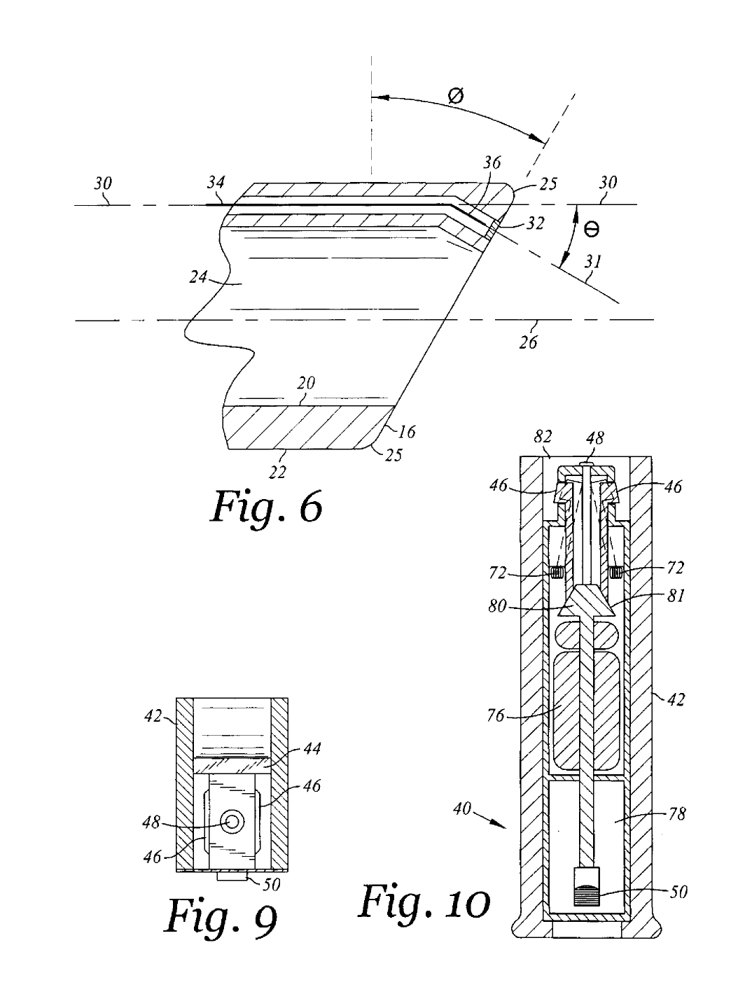

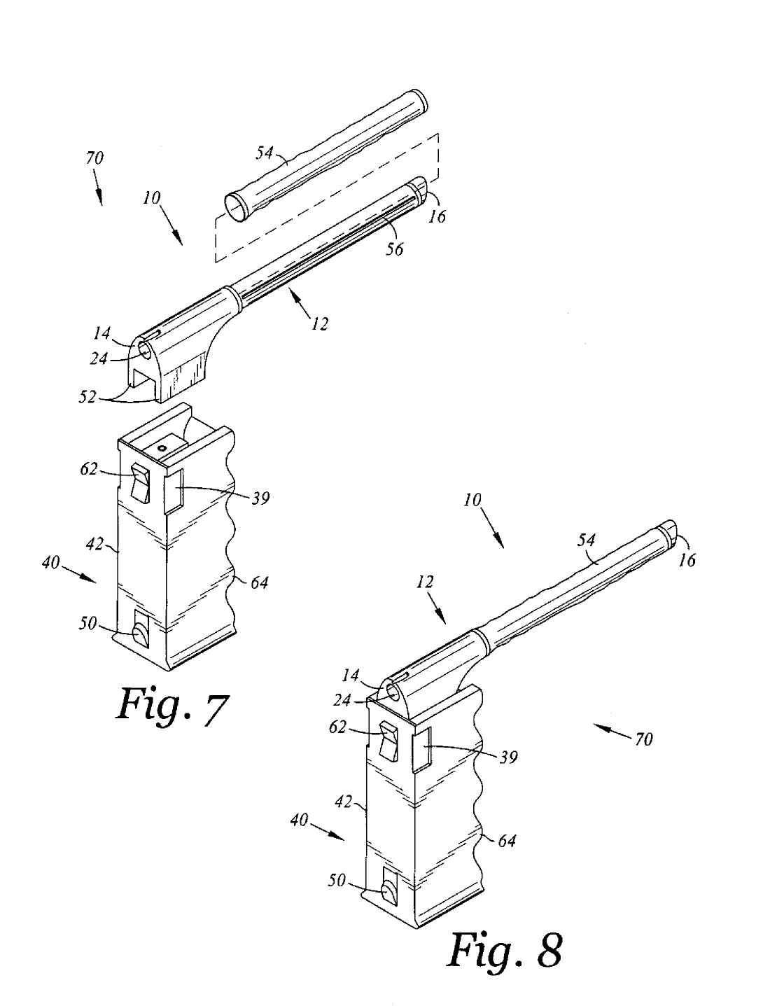

The longitudinal channel 28 extends along an optical axis 30. In one embodiment, the optical axis 30 is substantially linear and substantially parallel to the sampling channel axis 26. In another embodiment, and as best depicted in FIGS. 5 and 6, the optical axis 30 includes an angular offset 31 adjacent the tube second end 16. More specifically, the optical axis 30 is offset towards the sampling channel axis 26 by an angle 0. In one particular embodiment, the angular offset 31 is approximately 30 degrees; however, it is understood that the magnitude of the angular offset 31 may vary.

In the embodiment illustrated in FIG. 6, the angular offset 31 of the longitudinal channel 28 is substantially orthogonal to the tube second end 16. Accordingly, the second tube end 16 and an axis perpendicular to the sample channel axis 26 define an angle φ. Furthermore, as depicted in FIGS. 5 and 6, the longitudinal channel 28 is located above the sample channel 24. Therefore, the optical axis 30 extends downwardly adjacent the tube second end 16. However, it is understood that the longitudinal channel 28 may be below the sample channel 24. In this manner, the optical axis 30 may extend upwardly toward the sampling channel axis 26.

The angular offset 31 of the optical axis 30 is operative to direct the field of view of the fiber optic viewing device 34 toward the area of the body cavity 4 that is under examination and interfaces with the sampling tool 68. In this manner, the angular offset 31 directs the image receiving end 36 of the fiber optic viewing device 34 towards the examination area. Consequently, the individual performing the examination will be able to focus in on the examined area while still obtaining view of the sampling tool 68 interfacing with the patient 2. Although the angular offset 31 of the optical axis 30 illustrated in FIG. 6 includes a rather sharp curvature, it is understood that the angular offset 31 may include a smoother curvature. In other words, the angular offset 31 may be more gradual than the embodiment depicted in FIG. 6.

The medical examining device 10 further includes a translucent lens 32 positioned adjacent the longitudinal channel 28 adjacent the tube second end 16. According to one embodiment, the translucent lens 32 closes off the longitudinal channel 28 adjacent the tube second end 16. The translucent lens 32 is configured to allow light to pass therethrough. It is understood that the translucent lens 32 may be configured to allow varying amounts of light to pass therethrough. In other words, not all light entering the translucent lens 32 will necessarily pass through the translucent lens 32; some light may be reflected off the lens 32. The translucent lens 32 is also disposed in optical communication with the longitudinal channel 28. In this manner, light may pass through the translucent lens 32 and enter the longitudinal channel 28.

According to one embodiment, the fiber optic viewing device 34 is removably insertable within the longitudinal channel 28 during a medical examination. Therefore, the fiber optic viewing device 34 may be inserted into the longitudinal channel 28 for examining a patient 2. When the examination is complete, the fiber optic viewing device 34 may be removed from the longitudinal channel 28.

In order to insert the fiber optic viewing device 34 into the longitudinal channel 28, the image receiving end 36 is inserted into the longitudinal channel 28 to dispose the fiber optic image receiving end 36 adjacent to and in optical communication with the translucent lens 32. In this manner, light passing through the translucent lens 32 may enter the fiber optic image receiving end 36 for display on the display screen 38. The fiber optic viewing device 34 may substantially extend along the length of the longitudinal channel 28 (i.e., between the translucent lens 32 and the opening 29) when the fiber optic viewing device 34 is inserted therein.

The fiber optic viewing device 34 may be temporarily disposed within the longitudinal channel 28 during a medical examination of a first patient. After the examination of the first patient, the fiber optic viewing device 34 may be removed from the longitudinal channel 28, and inserted into the longitudinal channel 28 of a new sample tube 12 for an examination on a second patient. In this manner, the longitudinal channel 28 provides a protective barrier between the patient being examined and the fiber optic viewing device 34. The fiber optic viewing device 34 is sufficiently inserted into the longitudinal channel 28 to reduce or eliminate the need to sanitize the fiber optic viewing device 34 between uses on different patients. In other words, the medical examining device 10 may be disposable and intended for use on a single patient.

Visual examination of the body cavity 4 may necessitate illumination of the examined area. Therefore, one embodiment of the medical examining device 10 is configured to communicate light from a light source and emit the light at the tube second end 16. Accordingly, the medical examining device 10 includes a light receiving surface 60 disposable in optical communication with the light source to receive light therefrom. The light received from the light source is communicated along the medical examining device 10 and is emitted at the tube second end 16 to illuminate an examination area 66, as depicted in FIG. 12. Therefore, it may be desirable to form the medical examining device 10 out of a translucent material, such as glass or plastic. Furthermore, it may be desirable to include a reflective coating along portions of the medical examining device between the light receiving surface 60 and the tube second end 16 to mitigate emission of light at areas other than the tube second end 16.

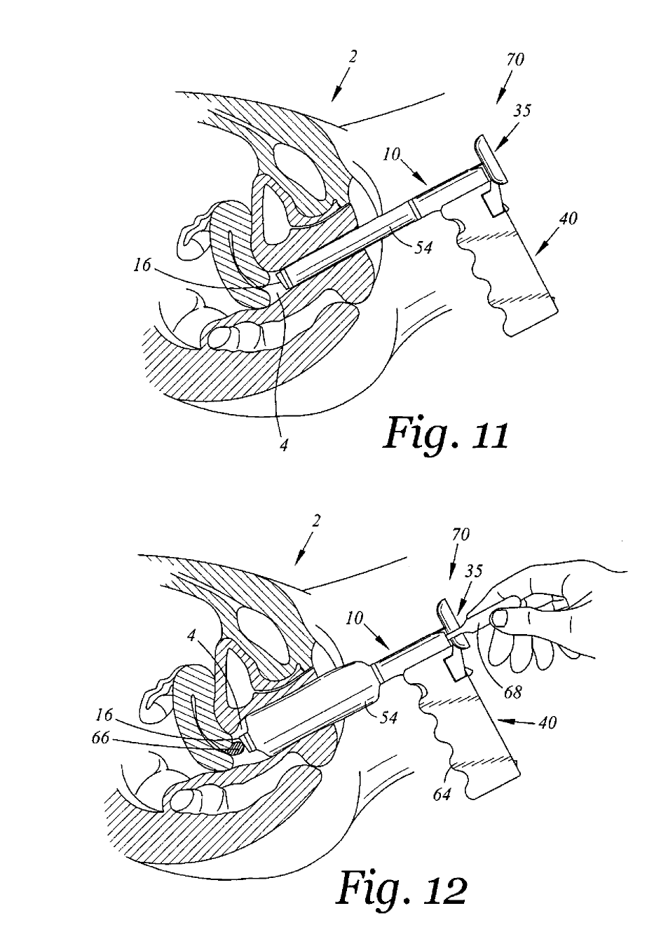

Furthermore, it is understood that proper examination of a body cavity 4 may require opening or spreading of the body cavity 4. Accordingly, one aspect of the present invention is directed toward an inflatable cuff 54 connected to the sample tube 12. The inflatable cuff 54 may be in a deflated state when the medical examining device 10 is inserted or removed from the patient's body cavity 4, as illustrated in FIG. 11. After insertion, the inflatable cuff 54 may be inflated to open or spread the body cavity 4, as illustrated in FIG. 12. The inflatable cuff 54 is formed of a flexible material to accommodate inflation and deflation thereof. Latex is a flexible material that was commonly used in the medical profession. However, Latex has been known to cause allergic reactions by patients. Therefore, other flexible materials are readily used in the medical profession as latex substitutes. Exemplary of such latex substitutes is nitrile. Therefore, one embodiment of the inflatable cuff 54 is formed from nitrile; however, it is understood that other materials known by those skilled in the art may also be used.

The inflatable cuff 54 may be circumferentially disposed about the sample tube 12 and extend along a portion of the sample tube 12. The sample tube 12 may include one or more fluid channels 56 disposed therein for providing fluid to the inflatable cuff 54. The fluid channels 56 may be in fluid communication with the inflatable cuff 54 and fluidly connectable to a fluid source to provide fluid to the inflatable cuff 54. The fluid channel 56 includes a fluid port 58 that is fluidly connectable to the fluid source. In one embodiment, the inflatable cuff 54 is filled with a gaseous fluid. For instance, the fluid channel 56 may be connected to a pressurized air source to inflate the inflatable cuff 54. However, in another embodiment, the inflatable cuff 54 is filled with a liquid fluid.

The medical examining device 10 may be used in connection with a central unit 40 to define a medical examining assembly 70. The central unit 40 may be used to provide light and/or fluid to the medical examining device 10. The central unit 40 may also provide a grip or handle for holding the medical examining device 10 during a medical examination. In one embodiment, the medical examining device 10 is detachably connected to the central unit 40. In this manner, one medical examining device 10 may be connected to the central unit 40 for examining a first patient. After the first patient's examination, the medical examining device 10 may be detached from the central unit 40. Afterwards, another medical examining device 10 may be connected to the central unit 40 for examination of a second patient. In this manner, a single central unit 40 may be used with several medical examining devices 10 to perform examinations on several patients.

The central unit 40 includes a central housing 42. In one embodiment, the central housing 42 includes an ergonomic contour 64 to facilitate gripping of the central unit 40. The central unit 40 is configured to be engageable with the medical examining device 10. According to one embodiment, the central unit 40 includes a pair of retention members 46 configured to engage with one or more retention flanges 52 extending from the sample tube 12. The retention members 46 are moveable between an engaged and a disengaged position relative to the central housing 42. When the medical examining device 10 is connected to the central unit 40, the retention members 46 are disposed in the engaged position to engage with the retention flanges 52. When a user desires to remove the medical examining device 10 from the central unit 40, the retention members 46 may be moved to the disengaged position to disengage from the retention flanges 52.

Referring now to FIG. 10, there is shown a sectional view of the central unit 40. In the embodiment illustrated, the retention members 46 are biased toward the engaged position by springs 72. In this manner, the springs 72 extend between the housing 42 and the retention members 46 to bias the retention members 46 into the engaged position. The retention flanges 52 of the medical examining device 10 may extend into a receiving cavity 82 formed within the central housing 42. The spring loaded retention latches 46 may press against the retention flanges 52 to secure the medical examining device 10 to the central unit 40.

The central unit 40 further includes a retention member separator 80 for moving the retention members 46 from the engaged position toward the disengaged position. In this regard, the retention member separator 80 may be disposed between the retention members 46 to move the retention members 46 into the disengaged position. As illustrated, the retention member separator 80 includes a conical cross section 81 to enable insertion of the retention member separator 80 between the retention members 46. The retention member separator 80 may be connected to a retention switch 50 to enable the user to move the retention member separator 80 for disengagement of the medical examining device 10 and the central unit 40.

Although the figures illustrate spring loaded retention latches 46 to secure the medical examining device 10 to the central unit 40, other fastening techniques known by those skilled in the art may also be used. For instance, the medical examining device 10 and the central unit 40 may include complimentary slots and grooves to facilitate engagement therebetween. The medical examining device 10 and the central unit 40 may also be configured to facilitate a press fit engagement therebetween. Furthermore, the medical examining device 10 and central unit 40 may include magnets configured to create a magnetic force to maintain the medical examining device 10 and central unit 40 in an engaged configuration. The forgoing engagement techniques are exemplary in nature only, and are not intended to limit the scope of the present invention.

In one embodiment, the central unit 40 includes a fluid source 76 disposed within the central housing 42. The fluid source 76 is in fluid communication with a fluid opening 48 for communicating fluid into and out of the fluid source 76. In other words, fluid may be transferred from the fluid source 76 to the medical examining device 10 via the fluid opening 48. Alternatively, if the fluid source requires re-filling, fluid may be inserted into the fluid source 76 via the fluid opening 48.

The fluid opening 48 is fluidly engageable with the fluid port 58 formed on the medical examining device 10 to facilitate fluid communication between the medical examining device 10 and the central unit 40. The fluid source 76 may include pressurized fluid or a fluid pump for supplying fluid to the medical examining device 10. Fluid may be communicated to the fluid source 76 to the medical examining device 10 for purposes for inflating the inflatable cuff 54. The central unit 40 may include an inflate/deflate switch 62 operatively connected to the fluid source 76 to allow a user to control inflation and deflation of the inflatable cuff 54.

According to another aspect of the invention, the central unit 40 includes a light source 44 for providing light to the medical examining device 10. When the medical examining device 10 is engaged with the central unit 40, the light receiving surface 60 formed on the medical examining device 10 is disposed in optical communication with the light source 44. In this manner, light emitted by the light source 44 is received by the light receiving surface 60 and is communicated along the length of the sample tube 12. The central unit 40 may also include a battery compartment 78 configured to receive batteries or other sources of power for operating the medical examining assembly 70.

The central unit 40 may additionally include a display mount 39 formed within the central housing 42 for engaging with the display screen 38 and maintaining the display screen 38 in a viewable position during the medical examination. The display screen 38 and display mount 39 may include complimentary slot and grooves, or magnetic connectors, or other connecting means known by those skilled in the art to facilitate engagement therebetween.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A medical examining device for use with a tissue sampling tool, the medical examining device comprising:
an elongate, hollow sample tube defining a tube first end and an opposing tube second end, the sample tube having a tube wall extending between the tube first end and the tube second end, the tube wall including a tube inner surface and a tube outer surface extending between the tube first end and the tube second end, the tube inner surface defining a central sampling channel extending along a sampling channel axis, the central sampling channel being sized and configured to longitudinally receive the sampling tool along the sampling channel axis;
a longitudinal channel extending partially through the tube wall from the tube first end towards the tube second end along an optical axis, the optical axis is angularly offset and extends toward the sampling channel axis adjacent the tube second end;
a translucent lens positioned in optical communication with the longitudinal channel adjacent the tube second end; and
a fiber optic viewing device disposed within the longitudinal channel and in optical communication with the translucent lens.

2. The medical device of claim 1 further comprising an inflatable cuff disposed about the sample tube.

3. The medical device of claim 2 further comprising at least one fluid channel formed within the tube wall, the fluid channel being in fluid communication with the inflatable cuff, and fluidly connectable to a fluid source.

4. The medical device of claim 2 wherein the inflatable cuff is circumferentially disposed about the sample tube.

5. The medical device of claim 2 wherein the inflatable cuff is formed of a nitrile material.

6. The medical device of claim 1 wherein the sample tube is formed of a translucent material.

7. The medical device of claim 1 further comprising a light receiving surface connected to the sample tube, the light receiving surface being disposable in optical communication with a light source, the light receiving surface being configured to receive light emitted by the light source.

8. A medical examining device for use with a tissue sampling tool, the medical examining device comprising:
an elongate, hollow sample tube defining a tube first end and an opposing tube second end, the sample tube having a tube wall extending between the tube first end and the tube second end, the tube wall including a tube inner surface and a tube outer surface extending between the tube first end and the tube second end, the tube inner surface defining a central sampling channel extending along a sampling channel axis, the central sampling channel being sized and configured to longitudinally receive the sampling tool along the sampling channel axis;
a longitudinal channel extending partially through the tube wall from the tube first end towards the tube second end along an optical axis, the optical axis extending toward the sampling channel axis adjacent the tube second end; and
a translucent lens positioned in optical communication with the longitudinal channel adjacent the tube second end.

9. The medical device of claim 8 further comprising an inflatable cuff disposed about the sample tube.

10. The medical device of claim 9 further comprising at least one fluid channel formed within the tube wall, the fluid channel being in fluid communication with the inflatable cuff, and fluidly connectable to a fluid source.

11. The medical device of claim 9 wherein the inflatable cuff is circumferentially disposed about the sample tube.

12. The medical device of claim 9 wherein the inflatable cuff is formed of a nitrile material.

13. The medical device of claim 8 wherein the sample tube is formed of a translucent material.

14. A method of using a medical examining device, the method comprising the steps of:
(a) providing a fiber optic viewing device;
(b) providing a first medical examining device including:
an elongate, hollow sample tube defining a tube first end and an opposing tube second end, the sample tube having a tube wall extending between the tube first end and the tube second end, the tube wall including a tube inner surface and a tube outer surface extending between the tube first end and the tube second end, the tube inner surface defining a central sampling channel extending along a sampling channel axis, the central sampling channel being sized and configured to longitudinally receive the sampling tool along the sampling channel axis;
a longitudinal channel extending partially through the tube wall from the tube first end towards the tube second end along an optical axis, the optical axis extending toward the sampling channel axis adjacent the tube second end; and
a translucent lens positioned in optical communication with the longitudinal channel adjacent the tube second end.
(c) inserting the fiber optic viewing device into the longitudinal channel of the first medical examining device to dispose the fiber optic viewing device in optical communication with the translucent lens of the first medical examining device;
(d) inserting the first medical examining device into a body cavity of a first patient;
(e) removing the first medical examining device from the body cavity of the first patient; and
(f) removing the fiber optic viewing device from the longitudinal channel of the first medical examining device.

15. The method of claim 14 wherein step (b) includes providing a first medical examining device including an inflatable cuff disposed about the sample tube.

16. The method of claim 15 wherein step (d) further includes inflating the inflatable cuff after the first medical examining device is inserted into the body cavity of the first patient.

17. The method of claim 14 further comprising the step of providing a second medical examining device, the second medical examining device including:
an elongate, hollow sample tube defining a tube first end and an opposing tube second end, the sample tube having a tube wall extending between the tube first end and the tube second end, the tube wall including a tube inner surface and a tube outer surface extending between the tube first end and the tube second end, the tube inner surface defining a central sampling channel extending along sampling channel axis, the central sampling channel being sized and configured to longitudinally receive the sampling tool along a sampling channel axis;
a longitudinal channel extending partially through the tube wall from the tube first end towards the tube second end along an optical axis, the optical axis extending toward the sample channel axis adjacent the tube second end; and
a translucent lens positioned in optical communication with the longitudinal channel adjacent the tube second end.

18. The method as recited in claim 17 further comprising the step of inserting the fiber optic viewing device into the longitudinal channel of the second medical examining device to dispose the fiber optic viewing device in optical communication with the translucent lens of the second medical examining device.

19. The method as recited in claim 18 further comprising the step of inserting the second medical examining device into a body cavity of a second patient.
